SEQUENCE LISTING (2 sequences)

SEQ ID NO: 1 - DNA, 20 bp, Bacillus stearothermophilus
aagtttaaagcgattgtaag

SEQ ID NO: 2 - DNA, 19 bp, Bacillus stearothermophilus
ctatttaatatgttacagc

(19) United States
(12) Patent Application Publication
(10) Pub. No.: US 2005/0186661 A1
(43) Pub. Date: Aug. 25, 2005

(54) Production of protease from Bacillus stearothermophilus F1

(76) Inventors: Raja Noor Zaliha Abd. Rahman, Selangor (MY); Abu Bakar Salleh, Selangor (MY); Mahiran Basri, Selangor (MY); Che Nyonya Abd. Razak, Selangor (MY); Kamaruzaman Ampon, Selangor (MY); Khatijah Mohd. Yusoff, Selangor (MY); Suhaili Abd. Hamid, Selangor (MY); Zhibiao Fu, Selangor (MY)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/062,089

(22) Filed: Feb. 18, 2005

(30) Foreign Application Priority Data
Feb. 20, 2004 (MY) 20040553

(51) Int. Cl. C07H 21/04; C12N 9/52; C12N 1/21; C12N 15/74

(52) U.S. Cl. 435/69.1; 435/220; 435/252.31; 435/471; 536/23.2; 435/252.33

(57) ABSTRACT

A biologically pure strain of Bacillus stearothermophilus F1 capable of Producing protease which is tolerant to any organic compound selected from the group leucine, cysteine, arginine, glycine, asparagine (BDH) and aspartic acid. Peptone iv derived from soybean was the best organic compound for the enzyme production. Sodium nitrate, ammonium salts and amino acids as sole nitrogen sources interfered with protease formation. In addition Bacteriocin-release-protein (BRP) system was used for the release of heterologous proteins from Escherichia coli into culture medium. The gene from the alkaline protease was cloned from Bacillus stearothermophilus F1 and the recombinant F1 protease was efficiently excreted into the culture medium using two vectors pTrcHis bearing the protease gene and pJL3 containing the BRPs. The recombinant enzyme was purified through single-step heat treatment at 70° C. for 3 hours at pH level from 8 to 10.

### FIELD OF INVENTION

The present invention relates to the fields of Enzyme technology and Microbiology. More particularly this invention relates to the production of protease from Bacillus stearothermophilus F1 from the culture medium for optimum production and a method for secretory expression of heat stable alkaline protease in Escherichia coli.

### BACKGROUND OF THE INVENTION

Proteases are degradative enzymes which catalyse the hydrolysis of peptides. Proteases play an important role in numerous biochemical reactions in living organisms, including degradation of proteins into amino acids and peptides for nutrients or during protein turn over processes, formation of spore and germination, coagulation, cascade reactions, post translation reactions, modulation of gene expression, enzyme modification and secretion of various protein enzymes biocatalyst (Suhartono et al., 1997).

Bacterial extracellular proteases have been the subject of vast literature report and have considerable value as a source of industrial enzymes (Priet, 1977). Microbial proteases dominate commercial applications, with a large market share taken by subtilisin proteases from Bacillus spp. for laundry detergent applications. Nutritional factors are known to affect the production of microbial enzymes. Therefore, considerable changes in fermentation media (or culture media) have to be made to increase the protease yield.

A major requirement for commercial applications is thermal stability, because thermal denaturation is a common cause of enzyme inactivation. There have been a number of recent efforts to improve the thermostability of enzymes on the basis of the currently limited knowledge of protein engineering.

An alternative method for obtaining enzymes to improve thermostability is to isolate enzyme from naturally occurring thermophilic organisms. However, the disadvantage of this approach is that it is impractical to produce large quantities of enzymes from such organisms, as yield may be low because of imprecise growth conditions. Furthermore, high-temperature fermentations may require special equipment. Therefore, the preferred method is to use gene technology to clone and express the thermophilic genes of interest in mesophilic organisms.

There have been few reports on the release of recombinant protease into the culture media of Escherichia Coli (E. coli). There are several advantages to a system that releases proteins in a regulated manner into the culture media. Among them are, purification of the protein of interest is simplified, the culture media provides a larger space for accumulation of the protein, and release of the protein will not result in cell death or lysis as often occurs in high-level cytoplasmic production of recombinant proteins.

### SUMMARY OF THE INVENTION

The invention discloses the maximum F1 protease production obtained by using organic nitrogen source and Bacteriocin-release-protein (BRP) system is used for large-scale production of F1 protease in E. coli.

One aspect of the present invention is therefore related to a method for producing F1 protease of Bacillus stearothermophilus F1 comprising the steps of:
(i) cultivating Bacillus stearothermophilus F1 in a BSM culture medium at a temperature not exceeding 80° C. at a pH level between 5.0-11, and
(ii) extracting the protease from said culture medium, wherein the BSM culture medium has been supplemented with 1% (w/v) of at least one organic nitrogen source selected from the group of peptone iv, soytone, corn steep liquor, beef extract, tryptone, casein, and gelatine.

Preferably, the BSM culture medium contains 0.5 g/l CaCl₂×2H₂O; 0.2 g/l K₂HPO₄; 0.5 g/l MgSO₄×7H₂O; 0.2 g/l KCl; and 0.1 g/l NaCl.

In a particularly preferred embodiment of the method of the present invention, organic nitrogen source is selected from peptone iv and/or soytone.

In another preferred embodiment of the method of the present invention, furthermore 0.5% raffinose is added.

In yet another preferred embodiment of the method of the present invention, furthermore 4.5 mM of calcium and/or strontium is added.

Another aspect of the present invention then relates to a method for recombinantly producing F1 protease of Bacillus stearothermophilus F1 in E. coli comprising the steps of:
(a) providing an expression vector comprising one or more DNA sequences encoding for one or more F1 protease enzyme gene products;
(b) transforming said E. coli host bacterium containing the bacteriocin-release-protein (BRP) system with the said expression vector;
(c) growing said transformed bacterium in a suitable growth medium; and
(d) isolating said protease from E. coli growth medium.

Preferably, the concentration of isopropyl-1-thio-D-galactopyranoside (IPTG) for induction of the BRP system is about 40 μM. “About” shall herein mean a given value±5%.

Yet another aspect of the present invention relates to a method for recombinantly producing F1 protease in E. coli as above, furthermore comprising the step of: (e) analysing said F1 protease activity from said culture medium.

In a particularly preferred embodiment of the method for recombinantly producing F1 protease in E. coli as above, the protease activity is analysed using azocasein as a substrate.

Preferably, isolating said protease from E. coli growth medium comprises a single-step purification comprising the step of freeze drying and heating the crude culture medium at 70° C. for 3 hours. More preferably, the F1 protease gene is present in the expression vector pTrcHis.

Another aspect of the present invention then relates to a protease, produced according to a method as above. Another aspect of the present invention then relates to a precursor protease produced according to a method as above, wherein said protease has a molecular mass of 30 kDa. Another aspect of the present invention then relates to a mature protease produced according to a method as above, wherein the protease has a molecular mass of 27 kDa.

Finally, the present invention relates to a use of a protease produced according to a method as above in detergent industries.

### DESCRIPTION OF THE FIGURES

The invention will now be described in detail with referral to a preferred embodiment and to the drawings in which:

FIG. 1 is a graph showing effect of different concentrations of peptone iv on protease production. Bars correspond to standard deviation.

FIG. 2 is a graph showing effect of additional carbon sources on protease production in peptone iv medium. Bars correspond to standard deviation. *: peptone iv medium without additional carbon source.

FIG. 3 is a graph showing effect of carbon source on protease production in sodium nitrate medium. Bars correspond to standard deviation. *: peptone iv medium without additional carbon source.

FIG. 4 is a graph showing effect of calcium concentration on protease production in peptone medium. Bars correspond to standard deviation.

FIG. 5 is a graph showing effect of metals on protease production in peptone iv medium. Bars correspond to standard deviation. Control denotes a culture without any metal ions.

FIG. 6 is SDS-PAGE analysis of Bacillus stearothermophilus F1 protease excretion by the E coli XL1-Blue cells harboring the two plasmids (pJL3 and recombinant pTrcHis). Note: Lane 1, standard marker. Lane 2, the total cell lysate from 24 hour cultivation. Lane 3, the total cell lysate from 30 hour cultivation. Lane 4, the total cell lysate from 48 hour cultivation. Lane 5, the total cell lysate from 72 hour cultivation. Lane 6, the medium concentrate from 24 hour cultivation cells. Lane 7, the medium concentrate from 30 hour cultivation cells. Lane 8, the medium concentrate from 48 hour cultivation cells. Lane 9, the medium concentrate from 72 hour cultivation cells. The E. coli cells grow in LB broth in the presence of 40 μM IPTG.

FIG. 7 is SDS-polyacrylamide gel electrophoresis (12%, w/v) of the purified protease. Note: Lane 1, molecular mass markers (in kilodaltons); Lane 2, crude enzyme; Lane 3: purified enzyme.

FIG. 8 is a graph showing Effect of different temperatures on enzyme stability. Note: The enzyme in Tris-HCl-2 mM Ca²⁺ (pH 9.0) was pre-incubated for various times at 85° C. (♦), 90° C. (▪) and 95° C. (▴). Error bars represent the standard deviations of triplicate determinations.

FIG. 9 is a graph showing Effect of pH on enzyme stability. Note: The enzyme solution was preincubated at 70° C. for 30 minutes (▪) and 24 hour (▴). The residual activity was measured at pH 9. Error bars represent the standard deviations of triplicate determinations.

FIG. 10 is a graph showing effect of inhibitors on protease activity. Note: The enzyme was incubated with inhibitors for 30 minutes at room temperature. The remaining activity was measured against 0.8% azocasein in 0.1 M Tris-HCl-2 mM Ca²⁺, pH 9.0 buffer at 70° C. * indicates the concentration of inhibitors used,—*:1 mM; **:2.5 mM; ***:10 mM. Error bars represent the standard deviations of triplicate determinations.

### DESCRIPTION OF THE SEQUENCES

SEQ ID NO:1 is a primer used according to the subject invention.

SEQ ID NO:2 is a primer used according to the subject invention.

### DESCRIPTION OF THE INVENTION

A thermophilic Bacillus stearothermophilus F1 that produced an extremely thermostable alkaline protease (F1 protease) was isolated by (Rahman et al., 1994) (Accession No: AY028651) This strain is able to grow at high temperatures up to 80° C. and within a broad pH range of 5.0 to 11.0.

The alkaline protease produced by B. stearothermophilus F1 had remarkable characteristics that can be marketable as industrial enzymes (Rahman et al., 1994). Among them, was its stability at 80° C. within the period tested (10 h) and a half-life of about 4 hour, 25 minutes and 8 minutes at 85° C., 90° C. and 95° C., respectively (Rahman et al., 1994). In addition, the thermostability of F1 protease was better than thermolysin and subtilisin (Salleh et al., 1997).

In liquid medium, the F1 protease was secreted after 12 hour incubation at 60° C., which corresponds to the mid-exponential growth phase of the bacteria, with a maximum production after 24 hours.

This invention discloses a composition of culture medium for optimum production of protease from B. stearothermophilus F1, the use of bacteriocin-release-protein (BRP) in the release of recombinant B. stearothermophilus F1 protease into an Escherichia coli culture medium and single-step purification of the recombinant F1 protease to homogeneity. BRP is a small lipoprotein with 28 amino acid residues, which is produced as a precursor with a signal peptide and secreted across the cytoplasmic membrane, where it is N-acetylated and inserted into the outer membrane. In the outer membrane, BRP can activate the detergent-resistant phospholipase A, resulting in the formation of permeable zones in the cell envelope, through which protein can pass and be released into the culture medium. Controlled expression of the BRP is used for the release of protease.

Nutritional Factors Affecting Protease Production

1 ml seed culture of B. stearothermophilus F1 in tripticase soy broth (TSB) (Difco, Sparks, USA) was inoculated into 50 ml of culture medium and incubated at 60° C. with shaking at 100 rpm (Hotech horizontal shaker, Taiwan) for 24 hour. The culture medium (BSM) consists of a basal salts solution containing (g/l): CaCl₂ 2H₂O, 0.5; K₂HPO₄, 0.2; MgSO₄ 7H₂O, 0.5; KCl, 0.2 and NaCl, 0.1.

The nitrogen sources were divided into three groups, which referred to an organic nitrogen sources, inorganic nitrogen sources and amino acids. The followings organic nitrogen sources were added to BSM at 1% (w/v): peptone iv (Sigma, St. Louis, USA), soytone (Difco, Sparks, USA), corn steep liquor (Difco, Sparks, USA), beef extract (Difco, Sparks, USA), tryptone (Difco, Sparks, USA), yeast extract (BBL, Franklin Lakes, USA), peptone 1 (peptone from meat, Sigma, St. Louis, USA), polypeptone (BBL, Franklin Lakes, USA), peptone (Microbiologie, La Courneuve, France), casein (Sigma, St. Louis, USA), urea (BDH, Poole, England), casamino acids (Difco, Sparks, USA) and gelatine (Merck, Darmstadt, Germany).

The inorganic nitrogen sources (1%, w/v) used was sodium nitrate, ammonium sulphate, and ammonium nitrate. For the effect of amino acids, 1% (w/v) of the following: leucine (Sigma, St. Louis, USA), cysteine (Sigma, St. Louis, USA), arginine (Sigma, St. Louis, USA), glycine (Sigma, St. Louis, USA), asparagine (BDH, Poole, England), and aspartic acid (Sigma, St. Louis, USA) were added to the BSM. When inorganic nitrogen source and amino acid were used as sole source of nitrogen, glycerol (0.5%) was added to the BSM. Supplementation of protein medium (peptone iv medium) with inorganic nitrogen source was carried out by the addition of sodium nitrate (1% and 0.5%) (w/v) to 1% (w/v) peptone iv medium (BSM+peptone iv).

For the effect of carbon sources, the following were added to the peptone iv medium at 0.5% (w/v); mannitol, glucose, sucrose, sorbitol, glycerol, starch, maltose, raffinose, galactose, ethanol, mannose, melibiose and trehalose. For the effect of carbon source in inorganic nitrogen medium, sodium nitrate (1%, w/v) was used as the sole source of nitrogen. All carbon sources were separately sterilized by membrane filtration (0.25 μm membrane filter).

The effect of calcium (chloride form) on protease production was determined by using calcium free medium (peptone iv medium without CaCl₂.2H₂O) or peptone iv medium containing various concentration of calcium. When the effects of other metals were being considered, calcium in peptone iv medium was replaced by other metals at specified concentration (4.5 mM). Initial pH of the medium was adjusted to pH 10.0. Bacterial growth was monitored by measuring the OD (optical density) at 679 nm.

After the appropriate incubation period, each B. stearothermophilus F1 culture was centrifuged at 15,000×g for 20 minutes at 4° C. The clear supernatant was assayed for proteolytic activity as previously described (Rahman et al., 1994). One unit (U) of enzyme activity was defined as the amount of activity that produces a change of absorbance (0.001 per minute) at 450 nm at 70° C. under the standard assay conditions.

Almost all of the organic compounds tested supported good growth. However, enzyme production was detected only with peptone iv, soytone, corn steep liquor, casein, gelatine and beef extract. Maximum protease production was obtained when peptone iv from soybean was used as the sole nitrogen and carbon sources (Table 1).

TABLE 1
Effect of nitrogen sources on protease production.

| Nitrogen Source (1%) | Protease Activity (U/ml) | Growth (OD 679) |
|---|---|---|
| Peptone iv | 9520 ± 115 | 0.70 ± 0.01 |
| Soytone | 6560 ± 17 | 0.68 ± 0.03 |
| Corn steep liquor | 1022 ± 13 | 0.60 ± 0.05 |
| Gelatine | 860 ± 40 | 0.55 ± 0.01 |
| Casein | 730 ± 62 | 0.48 ± 0.04 |
| Beef extract | 690 ± 40 | 0.58 ± 0.02 |
| Peptone I | 0 | 0.56 ± 0.07 |
| Polypeptone | 0 | 0.60 ± 0.01 |
| Tryptone | 0 | 0.58 ± 0.03 |
| Yeast extract | 0 | 0.50 ± 0.01 |
| Urea | 50 ± 10 | 0.20 ± 0.07 |
| Casimino acid | 0 | 0.10 ± 0.01 |
| | | 0.45 ± 0.05 |
| Sodium nitrate | 1963 ± 47 | |
| Ammonium sulfate | 45 ± 13 | 0.29 ± 0.08 |
| Ammonium nitrate | 60 ± 10 | 0.23 ± 0.09 |
| Asparagine | 433 ± 61 | 0.20 ± 0.06 |
| Aspartic | 217 ± 76 | 0.28 ± 0.08 |
| Leucine | 0 | 0.15 ± 0.05 |
| Cysteine | 0 | 0.10 ± 0.03 |
| Arginine | 0 | 0.18 ± 0.04 |
| Glycine | 0 | 0.10 ± 0.07 |

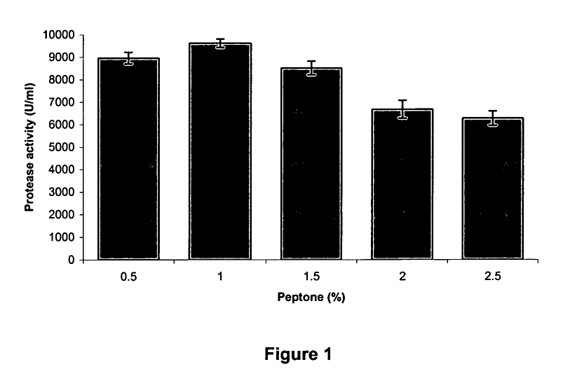

The effect of peptone iv on protease production by B. stearothermophilus F1 concentration as shown in FIG. 1. Peptone at 1% gave the best yield. Increasing the concentration of peptone iv reduced the protease production suggesting that, the excess protein resulted in the accumulation of nitrogenous catabolites such as amino acids and ammonia, which in turn reduced the protease synthesis. Growth and protease production by B. stearothermophilus F1 were also detected in a non-protein medium such as sodium nitrate (Table 1). However, employing inorganic compounds as sole nitrogen source significantly affected the growth as well as protease production. Protease production in sodium nitrate medium (sodium nitrate+BSM+Glycerol) was only about 11% of that with peptone iv, while ammonium compound completely inhibited the production. The presence of ammonium had been reported to interfere and significantly reduce protease production for other Bacillus and other bacteria species (Johnvesy and Naik, 2001, Bascaran et al., 1990, Jensen et al., 1980).

The use of amino acid as nitrogen source repressed and reduced both the protease production and growth of B. stearothermophilus F1. Very little growth and protease production was observed when the following amino acids was used as the sole nitrogen source; leucine, cysteine, arginine and glycine (Table 1). Asparagine and aspartic acid gave only 2 to 4% protease activity compared to control (peptone iv). The possible synergistic effect produced by combining both the best organic and inorganic nitrogen source was also examined. The result showed that the protease production was reduced slightly and the inhibitory effect was concentration dependent (Table 2). The combination however did not significantly alter the cell growth.

TABLE 2
Effect of sodium nitrate addition in peptone IV medium on
protease production.

| | Concentration | Protease | Growth |
|---|---|---|---|
| Nitrogen Source | % (w/v) | Activity (U/ml) | OD 679 |
| Peptone:NaNO₃ | 1:0 | 9530 ± 10 | 0.70 ± 0.01 |
| Peptone:NaNO₃ | 1:0.5 | 8185 ± 26 | 0.68 ± 0.020 |
| Peptone:NaNO₃ | 1:1 | 7065 ± 20 | 0.65 ± 0.01 |
| Peptone:NaNO₃ | 0.5:0.5 | 6975 ± 10 | 0.65 ± 0.020 |

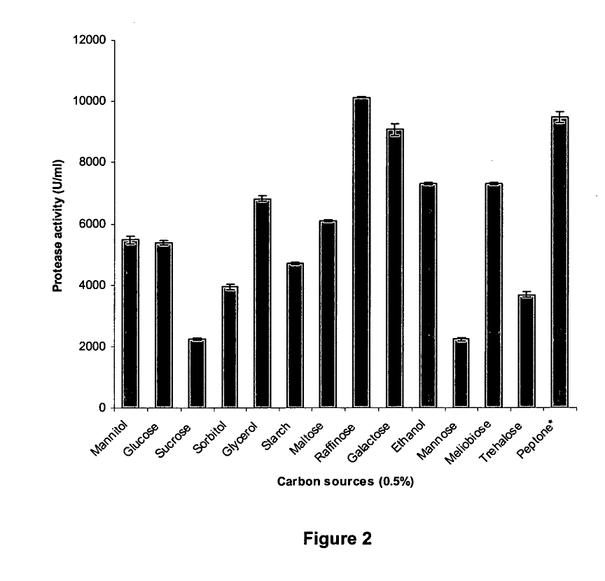

The presence of additional carbon source in peptone iv medium resulted in reduction in protease production by B. stearothermophilus F1 with the exception of raffinose (FIG. 2). The addition of these sources, however, slightly increased the growth of the bacterium. B. stearothermophilus F1 exhibited growth OD reading in the range of 0.68 to 0.82 when various carbon sources were added in peptone iv medium. Addition of raffinose to the protein medium slightly increased (6%) the protease production. However, Galactose showed a slight reduction (5%) in activity. Other carbon sources such as starch, sorbitol, trehalose, sucrose, and mannose showed low level of activity for protease production. These carbon sources reduce more than 50% of the protease production of B. stearothermophilus F1. The addition of maltose, glucose and mannitol caused a reduction of 37%, 43%, and 44% of protease production.

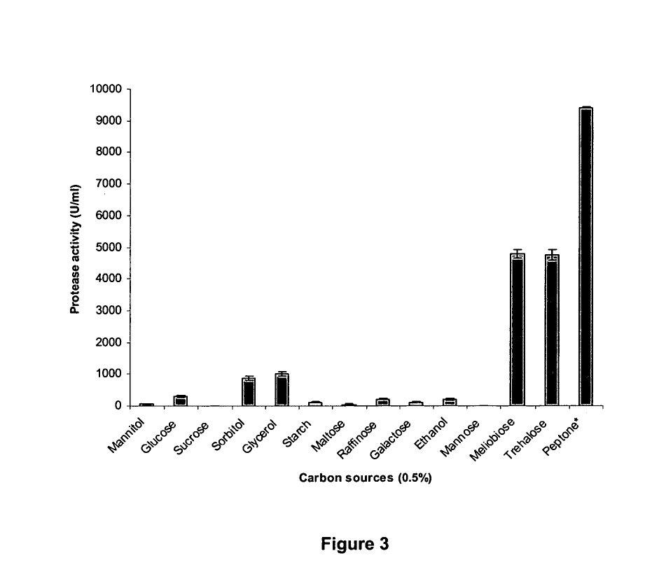

FIG. 3 shows the effect of various carbon sources in sodium nitrate medium for the protease production by B. stearothermophilus F1. The growth of the bacterium in the presence of these carbon sources showed no significant variation among them exhibiting an OD reading of around 0.40.

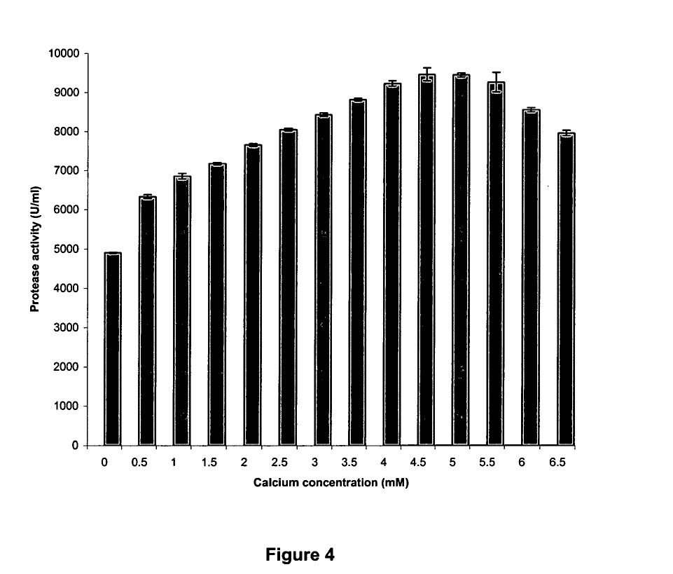

All of the carbon sources tested, only meliobiose and trehalose stimulated the protease production. However, the protease yields obtained were only 50% of that in peptone iv medium. The other carbon sources drastically reduced the production (glucose, sorbitol, glycerol, starch, maltose, raffinose, galactose, and ethanol) or completely inhibited it (sucrose, mannose). Calcium was not required for protease formation but by adding calcium in the peptone iv medium enhanced the yield. By increasing the concentration of calcium to 4.5 mM enhanced the production of protease, but by increasing the concentration of calcium further it will decrease the enzyme production (FIG. 4).

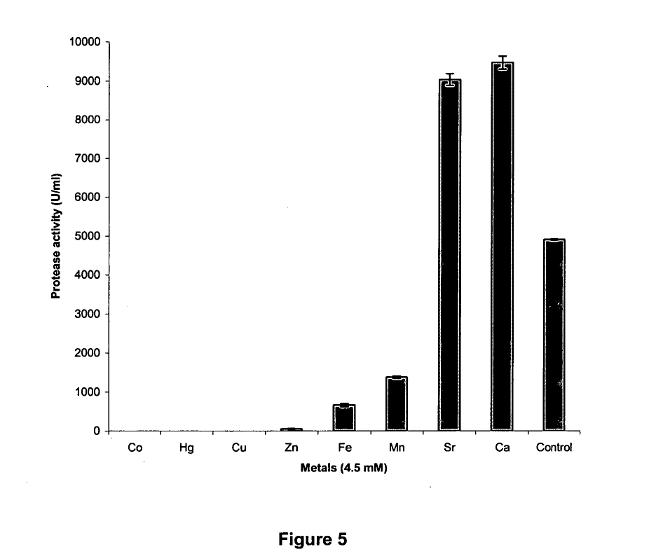

Besides calcium, strontium was equally effective as an inducer for the enzyme production (FIG. 5). Replacing the ions with Co²⁺, Cu²⁺ and Hg²⁺ completely inhibited the growth. The presence of Zn²⁺ ion in the medium resulted in significant reduction in both growth and production of the enzyme. Addition of Mn²⁺ and Fe²⁺ did not affect the growth significantly but decreased the enzyme activity by 85.5% and 93% respectively.

Each experiment is done in triplicates and standard deviation was determined from Microsoft Excel program. In this study the production of the alkaline protease by B. stearothermophilus F1 has greatly influence by the composition of culture medium. The growth of was very low when the bacterium was grown in the presence of inorganic nitrogen sources (ammonium sulfate and ammonium nitrate) and amino acids (Table 1).

Inorganic nitrogen sources significantly affected the growth of protease as well as protease production by B. stearothermophilus F1. Ammonium salts and amino acids interfered with the protease production whenever they are added to the production media. Most carbon source added in the peptone iv medium acted as carbon catabolite repressor and thus could be omitted from an economical point of view. A similar type of glucose repression found in B. stearothermophilus F1 had been shown to be involved in the regulation of extracellular protease in other Bacillus spp. (Klimov et al., 1988, Freeze et al., 1979) and other bacteria (Gusek et al., 1988, Whooley et al., 1983).

Metals were not required for protease production by this bacterium; however the presence of calcium and strontium enhanced the enzyme. Calcium at 4.5 mM maximized the enzyme production by F1. It must be stated that calcium was not determined in the calcium free medium. Contamination may occur from the other salts added and the peptone, but this contribution would be in minute quantities. For those bacteria requiring calcium for protease production, the formation and stabilization of protease were reported to be dependent on the concentration of the free calcium ions (Sidler and Zuber, 1977).

The presence of phosphate buffer removed the free calcium ion from the medium, thus reducing the enzyme formation. Therefore, for these bacteria, omission or reduction of phosphate as a buffer component in the culture medium resulted in a higher yield of protease (Sidler and Zuber, 1977). For B. sterothermophilus F1, the presence of phosphate in the production medium did not significantly influence the protease production (data not shown). This further strengthened the fact that calcium was not needed for enzyme formation of this bacterium.

The above studies showed that maximum protease production at lab-scale was obtainable using organic nitrogen source.

Secretory Expression of Protease

Bacterial Strain and Plasmids.

B. stearothermophilus F1, which harbors the F1 protease, was isolated from decomposed oil palm branches. The bacterial strains used for all recombination and expression work were E. coli TOP 10 and XL1-Blue. pCR2.1-TOPO® (Invitrogen, USA) was used for recombinant work. Plasmid pTrcHis-TOPO® (Invitrogen, USA) was used for expression of the F1 protease. Plasmid pJL3 (Mobitech, Germany) coded the BRP and helps the F1 protease secretion.

Media and Growth Conditions

Bacillus stearothermophilus F1 was cultured in trypticase soy broth (TSB) at 60° C. in a horizontal shaker (150 rpm). E. coli strains harboring pCR2.1 or pTrcHis were grown in Luria broth (LB) containing tryptone (10 g/ml), yeast extract (5 g/ml), NaCl (10 g/ml) and ampicillin (50 μg/ml). Cells harboring pJL3 and recombinant pTrcHis were grown in LB with ampicillin (70 μg/ml) and chloramphenicol (34 μg/ml). When solid media were required, Bacto agar was added to liquid medium to a final concentration of 1.5% (w/v).

DNA Manipulations

Bacillus stearothermophilus F1 chromosomal DNA was isolated by using modified method of Sambrook et al (A laboratory manual, second ed., Cold Spring Harbor Laboratory. Cold Spring Harbor, N.Y., 1989). Small-scale plasmid DNA was isolated by High Pure Plasmid Isolation Kit (Boehringer, Germany) according to the manufacturer's instructions. DNA fragments were purified from agarose gels by using QIAquick Gel Extraction Kit as recommended (Qiagen, Germany). All restriction and modifying enzymes were purchased from Promega (Madison, Wis.). All other chemicals and reagents were purchased from Sigma Chemical Co. (St. Louis, Mo.) unless otherwise specified.

Expression of F1 Protease in E. coli and SDS-PAGE Analysis

The open reading frame of F1 protease gene (Accession No.: AY028615 in Genbank) was cloned into pTrcHis-TOPO® plasmid by Polymerase Chain Reaction (PCR) at the 5′ and 3′ ends of the F1 gene. Primer 1 (5′-MGTTTAAAGCGATTGTMG 3′; SEQ ID NO:1) and primer 2 (5′ CTATTTMTATGTTACAGC 3′; SEQ ID NO:2) were annealed to generate a PCR product from B. stearothermophilus F1 chromosomal DNA. PCR was performed in a thermocycler (Gene Amp PCR system 2400, Perkin Elmer, Foster, Calif.) with the following conditions: [95° C./5 minutes]×1+[95° C./1 minute, 50° C./45 seconds, 72° C./1 minute]×30, uses Pfu polymerase (Stratagene, CA). After subcloning the PCR fragment into pTrcHis-TOPO® plasmid by TOPO TA cloning, the sequence of the insert was confirmed by sequencing (ABI Prism, model 310) and found to be in frame and identical with F1 sequence.

The BRP expression vector pJL3 was co-transformed into E. coli XL1-Blue strain with the F1 protease expression vector—recombinant pTrcHis-TOPO® by using standard calcium transformation procedures (Sambrook et al.) in a two-step process. The transformants were selected on LB plates containing chloramphenicol (34 μg/ml) and ampicillin (70 μg/ml) for pJL3/pTrcHis transformants.

E. coli XL1-Blue cells harbouring the expression plasmids were grown in 50 ml LB medium containing chloramphenicol (34 μg/ml) and ampicillin (70 μg/ml) overnight at 37° C., and was subcultured in 500 ml LB medium to grow to an optical density of 0.5-1 at 660 nm. After induction with 40 μM IPTG for 24, 30, 48, and 72 hour, aliquot (1 ml) of the induced cell culture was aliquot in a 1.5 ml microcentrifuge tube. The cell pellets were then resuspend in 200 μl of 2× sample buffer. The culture medium was prepared by acid precipitation with an equal volume of 20% (w/v) trichloroacetic acid (TCA). Precipitated protein was pelleted by centrifugation at 13,000×g for 10 minutes, and the supernatant was discarded.

The pellet was then washed with 70% ethanol, and after centrifuging, the supernatant was removed. The pellet was dried at room temperature for 10 minutes. Precipitated samples were then resuspended in 30 μl of 2×SDS sample buffer. The cell pellet sample was boiled for 10 minutes, while the medium pellet sample was boiled for 5 minutes before loading into the gel. One aliquot (10 μl) was loaded onto 12% SDS-polyacrylamide gel and electrophoresed at 30 mA for 1.5 hour. The gel was then stained with coomassie brilliant blue R-250.

Enzyme and Protein Assay

The enzymes are isolated from naturally occurring thermophilic organisms. The Measurement of protease activity using azocasein as the substrate is known in the art. Protein concentrations were determined using the standard curve of bovine γ-globulin.

Detection of Proteolytic Activity

Discontinuous native polyacrylamide gel [6%-10% (w/v)] electropheresis was used to separate the proteins. The proteins were stained with Coomassie brilliant blue R-250 (5 μg/ml) and the activity stain was detected directly on the other half of the gel by modifying the procedure of Arvidson and Wadstrom. The gel was overlaid with a solution of azocasein in molten agarose (equal volume of 0.8% azocasein in 0.02M Tris-HCl-2 mM CaCl₂, pH 9.0, with 2.5% molten agarose at 45° C.). The mixture was poured over the surface of the gel, incubated overnight at 55° C., and then immersed in a cold TCA (5%) solution. The appearance of a clear zone indicated the presence of protease activity.

Secretory Expression of the F1 protease in E. coli

Co-transformation of E. coli XL1-Blue with the recombinant pTrcHis and the BRP plasmid (pJL3) enabled the inventor to obtain positive clones, which expressed F1 protease and released it into the culture medium. Both vectors contain tandemly the E. coli lac promoter-operator system. The tandem promoter drives the expression of BRP and F1 protease and is regulated by E. coli lac repressor. The optimal concentration of isopropyl-1thio-D-galactopyranoside (IPTG) for BRP induction and F1 protease release was found to be about 40 μM. With lower concentration of IPTG, the E. coli cells harboring the plasmids grew to higher cell density; however, the amount of F1 protease released into culture medium was lower. Higher concentration of IPTG (100 μM) resulted in the retardation of cell growth as well as a lower yield of F1 protease released into the medium. When E. coli cells were grown in LB broth without IPTG, the cells grew well but almost no F1 protease was released into the medium.

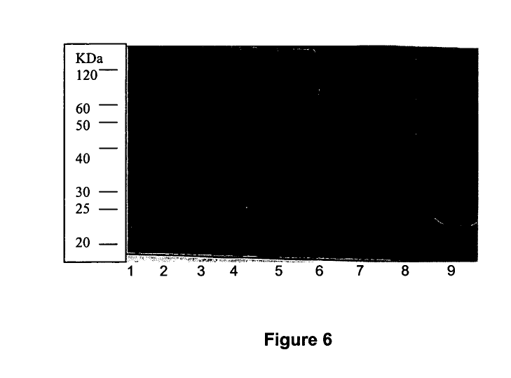

Examination of the SDS gels shown in FIG. 6 revealed that F1 protease is present in the culture medium, but is clearly not the only protein found there. However, comparison of the array of proteins found in the whole cell lysates with the proteins found in the culture medium demonstrated that F1 protease was relatively enriched in the medium. The amount of released F1 protease increases after prolonged incubation, with the culture containing the highest amount of F1 protease after 72 hour incubation (Lane 8).

However, the amount of F1 protease in the whole cell lysates was nearly the same from 24 hour to 72 hour (Lane 1-4). F1 proteases are supposed to have a molecular mass of 30 kDa for the precursor enzyme and 27 kDa for the mature enzyme.

Native discontinuous PAGE and proteolytic activity stain were performed to confirm the presence recombinant F1 protease. Cell-free culture supernatant, sonicated cell lysate and purified enzyme were analyzed.

Coomassie blue staining revealed a single protein band which exhibited proteolytic activity on agarose-azocasein agar (data not shown). Similar proteolytic activity was also observed on corresponding bands from cell free culture supernatant and sonicated cell lysate (data not shown).

Purification of the F1 Protease Through a Single Heat-Treatment Step

Heat treatment was used to precipitate the mesophilic E. coli proteins. The crude culture medium was concentrated 10-fold by freeze-drying and heat-treated in a hot water bath at 70° C. for 3 hour. Insoluble material was separated by centrifugation at 13,000×g for 10 min. The purified enzyme solution was lyophilized and stored at −80° C.

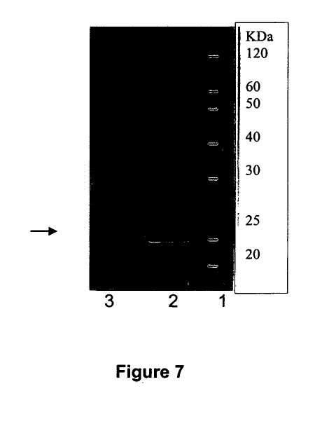

Wet cells (0.2 g) were pelleted from 100 ml of 24 hour induced cell culture. Application of heat treatment precipitated the mesophilic E. coli proteins, thus purifying the recombinant F1 protease. The specific activity of the enzyme also increases to 4.0-fold after heat treatment (Table 1). SDS-PAGE revealed one protein band, which migrates at approximately 27 kDa (FIG. 7). Staining analysis for protein and activity revealed a single protein band with protease activity as described above.

TABLE 3
Purification of recombinant F1 protease
from the culture supernatant of E. coli

| | | Total | Total | Specific | | Purification |
|---|---|---|---|---|---|---|
| | Volume | activity | protein | activity | Recov- | fold |
| Fraction | (ml) | (U^{a}) | (mg) | (U/mg) | ery | (%) |
| Crude | 100 | 11,000 | 24.6 | 447 | 100 | 1.0 |
| Heat | 100 | 10,900 | 6.1 | 1790 | 99 | 4.0 |
| treatment | | | | | | |

^{a}One unit of azocaseinase activity was defined as the enzyme activity equivalent to an absorbance change of 0.001 per minute at 70° C. under the standard assay condition.

Characterization of F1 Protease

The effects of temperature and pH on the hydrolysis of azocasein by recombinant F1 protease were studied by varying the pH from 5 to 12 and the temperature from 50 to 90π C. McIlvaine buffer (pH 5-7), Tris-HCl buffer (pH 7-9), Clark-Lub buffer (pH 9 and 10), carbonate buffer (pH 10 and 11), and phosphate buffer (pH 11 and 12) were employed to regulate the pH as described previously. For thermostability study, the enzyme was pre-incubated at pH 9 in 0.1 M Tris-HCl buffer, with 2 mM Ca²⁺ at different temperatures (85° C., 90° C., and 95° C.). Samples were taken out and immediately frozen prior to being assayed at intervals (1 hour at 85° C.; 15 minutes at 90° C.; 10 minutes at 95° C.). Patterns of inhibition were determined using inhibitors such as; PMSF (phenylmethanesulphonyl fluoride), EDTA (ethylenediaminetetraacetic acid), IM (iodoacetic acid), pepstatin and bestatin. These inhibitors were mixed with the F1 protease in 1:1 ratio (v/v), incubated at room temperature for 30 minutes, and the residual protease activity was then assayed using azocasein [0.8% (w/v) dissolved in 0.1 mM Tris-HCl plus 2 mM CaCl₂, pH 9.0].

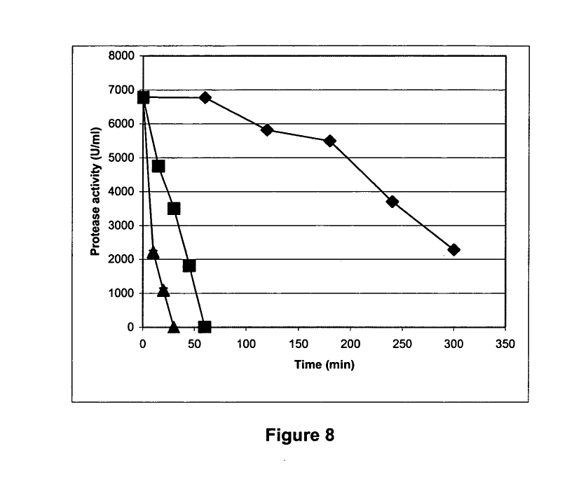

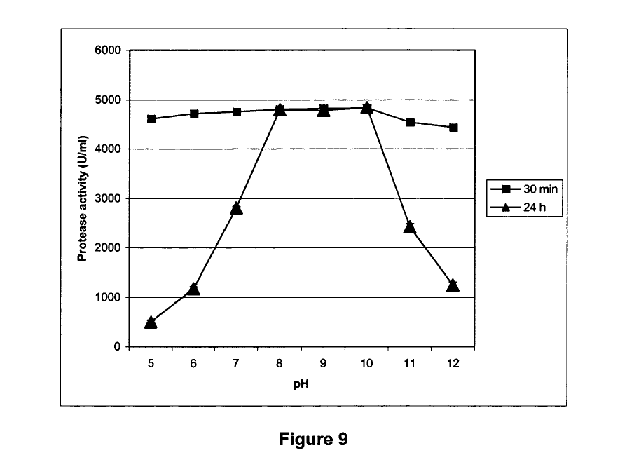

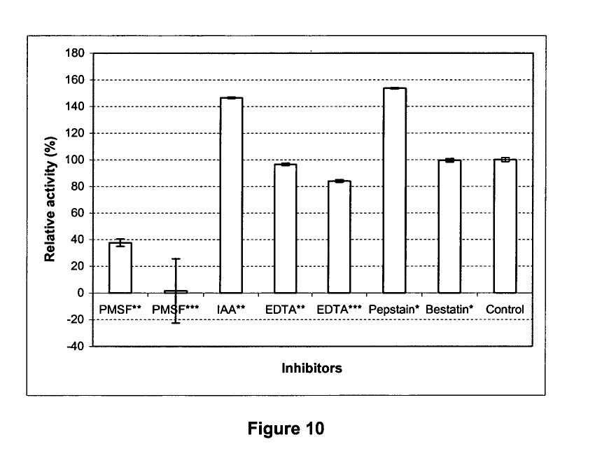

The characterization of the native F1 protease is known. The enzyme was optimally active at pH 9.0 and a half-life of 4 hour at 85° C., 25 minutes at 90° C. The recombinant purified enzyme displayed almost similar characteristic to those of the native F1 protease, with a half-life of 3.5 hour at 85° C., 25 minutes at 90° C. (FIG. 8). The enzyme was stable in the pH range from 8.0 to 10.0 at 70° C. for 24 hour (FIG. 9) and was inhibited only by the serine protease inhibitor phenylmethylsulfonyl fluoride (PMSF) (FIG. 10).

The pCIoDF13-encoded BRP is widely used in industry and scientific institutions, for extracellular production of homologous as well as heterologous proteins by E. coli. Utilizing BRP system allowed the expression of the protein of interest into the culture supernatant fraction with a minimal amount of other contaminating proteins. This results in an easier purification procedure and may also prevent intracellular inclusion body formation. The wild-type BRP causes severe growth inhibition of strongly induced cells and even cell death. These harmful side effects could be reduced by the lowering the concentration of IPTG used for induction.

Bacteriocin-release-protein (BRP) system was used to extracellularly express the recombinant F1 protease in E. coli. In these experiments, F1 protease was produced at a level estimated to be about 1500 unit/ml after 24 hour induction and released into culture medium. The culture medium was collected and concentrated without rupturing the cells, and F1 protease was purified to homogeneity through a one-step heat-treatment procedure at 70° C. for 3 hour. The cell extracts can be heated to precipitate and digest E. coli proteins. The recombinant F1 protease obtained was comparable with the native F1 protease isolated from the Bacillus stearothermophilus F1. These results show that BRP system could be used for the large-scale production of this F1 protease, and the possibility of continuous culture of E. coli.

1. A method for producing F1 protease of Bacillus stearothermophilus F1 comprising the steps of:
(i) cultivating Bacillus stearothermophilus F1 in a BSM culture medium at a temperature not exceeding 80° C. at a pH level between 5.0-11, and
(ii) extracting the protease from said culture medium,
wherein the BSM culture medium has been supplemented with 1% (w/v) of at least one organic nitrogen source selected from the group consisting of peptone iv, soytone, corn steep liquor, beef extract, tryptone, casein, and gelatine.

2. The method for producing F1 protease according to claim 1, wherein said organic nitrogen source is selected from the group consisting of peptone iv and soytone.

3. The method for producing F1 protease according to claim 1, wherein the culture medium further comprises 0.5% raffinose.

4. The method for producing F1 protease according to claim 1, wherein the culture medium further comprises 4.5 mM of calcium or strontium, or both.

5. A method for recombinantly producing F1 protease of Bacillus stearothermophilus F1 in E. coli comprising the steps of:
(a) providing an expression vector comprising one or more DNA sequences encoding for one or more F1 protease enzyme gene products;
(b) transforming E. coli host bacterium containing bacteriocin-release-protein (BRP) system with said expression vector;
(c) growing said transformed bacterium in a suitable growth medium; and
(d) isolating said F1 protease from E. coli growth medium.

6. The method for recombinantly producing F1 protease in E. coli according to claim 5, further comprising the step of BRP induction using isopropyl-1-thio-D-glactopyranoside (IPTG), wherein the concentration of IPTG for inducing BRP is about 40 μM.

7. The method for recombinantly producing F1 protease in E. coli according to claim 5, further comprising the step of:
(e) analysing said F1 protease activity from said growth medium.

8. The method for recombinantly producing F1 protease in E. coli according to claim 7, wherein the protease activity is analysed using azocasein as a substrate.

9. The method for recombinantly producing F1 protease in E. coli according to claim 5, wherein isolating said protease from E. coli growth medium comprises a purification process comprising the step of freeze drying and heating the crude culture medium at 70° C. for 3 hours.

10. The method for recombinantly producing F1 protease in E. coli according to claim 5, wherein the expression vector is pTrcHis, which expresses the F1 protease gene.

11. Use of a protease produced according to a method according to any preceding claim in detergent industries.
